(19) United States
(12) Patent Application Publication
(10) Pub. No.: US 2021/0369498 A1
(43) Pub. Date: Dec. 2, 2021

(54) TEMPERATURE REGULATING APPARATUS FOR A PROSTHETIC LIMB

(71) Applicant: Costello Prosthetic Warmers LLC, Liverpool, NY (US)

(72) Inventor: Bryan Costello, Liverpool, NY (US)

(73) Assignee: Costello Prosthetic Warmers LLC, Liverpool, NY (US)

(21) Appl. No.: 17/236,643

(22) Filed: Apr. 21, 2021

Related U.S. Application Data
(60) Provisional application No. 63/030,346, filed on May 27, 2020.

(51) Int. Cl. A61F 7/02; A61F 2/80; A61F 7/00

(52) U.S. Cl. CPC A61F 7/02; A61F 2/80; A61F 7/007; A61F 2002/5056; A61F 2007/0225; A61F 2007/0285; A61F 2007/0051

(57) ABSTRACT

A temperature regulating socket apparatus for selectively heating or cooling a residual limb of an amputee is provided. The temperature regulating socket apparatus includes a socket having a brim, a distal end and a continuous side wall, all defining an open interior area for receiving the residual limb therein and a temperature regulating apparatus. The temperature regulating apparatus includes a thermal adjustment element embedded within the socket side wall; a temperature control unit operatively coupled to the thermal adjustment element; and a power supply operatively coupled to the temperature control unit. When the power supply is in its ON orientation the temperature control unit is powered so that the temperature control unit regulates the temperature of the thermal adjustment element to selectively heat or cool the residual limb within the socket.

### CROSS-REFERENCE TO RELATED APPLICATIONS

This application claims the benefit of U.S. Patent Application No. 63/030,346, filed May 27, 2020, the contents of which are hereby incorporated by reference in its entirety.

### FIELD OF THE INVENTION

This invention relates generally to temperature modification apparatuses and, more particularly, to a temperature modification apparatus for a prosthetic limb configured to selectively impart heating or cooling to the residual limb of an amputee.

### BACKGROUND OF THE INVENTION

Persons who have experienced the amputation of a limb, such as a leg or an arm, must care for the residual limb that remains, including addressing the discomfort that occurs when the residual limb gets hot or cold depending upon environmental conditions or physical activity. An amputee's residual limb may get cold due to decreased blood circulation or due to interaction with a prosthetic device that is pulled on over the residual limb.

Various devices and proposals have been introduced in an attempt to help thermally regulate an amputee's residual limb, such as wearing a tube sock. In U.S. Application Publication No. 2012/0004738, it was proposed that a warming sleeve include a heated wire powered by a rechargeable battery. Although presumably effective for its intended purpose, the proposed unregulated heated sleeve has the potential to burn the person wearing it as it is in direct contact with the residual limb. In addition, the warming sleeve has a closed lower end such that it cannot be worn while the amputee is actually wearing a prosthetic assembly, such as an artificial arm or leg. In other words, the heated sleeve does not allow portions of a prosthetic assembly to extend therethrough.

Therefore, it would be desirable to have a prosthetic limb heating/cooling apparatus wherein the thermal units are embedded within the socket, and wherein the socket defines an open top and open bottom such that a traditional prosthetic assembly may still be worn in a normal fashion. Further, it would be desirable to have a prosthetic limb heating/cooling apparatus configured to receive a prosthetic liner therein such that the liner, and by thermal conduction the residual limb of an amputee, is selectively thermally adjustable by the wearer.

### BRIEF SUMMARY OF THE INVENTION

In accordance with an aspect of the present invention, one exemplary embodiment may be directed towards a temperature regulating socket apparatus for selectively heating or cooling a residual limb of an amputee. The temperature regulating socket apparatus includes a socket having a brim, a distal end and a continuous side wall, all defining an open interior area for receiving the residual limb therein, and a temperature regulating apparatus. The temperature regulating apparatus includes a thermal adjustment element embedded within the socket side wall; a temperature control unit operatively coupled to the thermal adjustment element; and a power supply operatively coupled to the temperature control unit. When the power supply is in its ON orientation the temperature control unit is powered so that the temperature control unit regulates the temperature of the thermal adjustment element to selectively heat or cool the residual limb within the socket.

In accordance with a further aspect, the thermal adjustment element may comprise a flexible heating element having a base member configured to be positioned at the distal end of the socket and a plurality of individually spaced apart heating units extending outwardly from the base member. Each of the heating elements is configured to extend along a length of the side wall toward the brim. The temperature control unit may selectively adjustably supply an electric current received from the power supply to each of the heating elements whereby the heating elements heat the socket through resistive heating. The base member may also be dimensioned to encircle a mounting element located at the distal end of the socket, where the mounting element is configured to couple a prosthetic device to the socket.

In accordance with another aspect, the power supply may be a battery. The battery and the temperature control unit may also be contained within a common housing. The socket may then include a bracket configured to removably secure the housing to the socket. Additionally, the socket may be manufactured using casting or lamination, and may include carbon fiber or fiberglass.

In accordance with still another aspect of the present invention, the thermal adjustment element may comprise a socket tube having opposing first and second ends, wherein the socket tube defines a serpentine path about the side wall. The temperature control unit may then comprise a thermal exchanger fluidly coupled to the first and second ends of the socket tube, along with a pump. A heat exchange fluid may then be circulated through the socket tube and the thermal exchanger via the pump. The heat exchange fluid may be water.

In a further aspect, the thermal exchanger may include an exchange tube having an inlet end and an outlet end. The inlet end may be coupled to the first end of the socket tube via a first coupling while the outlet end may be coupled to the second end of the socket tube via a second coupling whereby the socket tube and the exchange tube form a closed loop circuit. The first and second couplings may be leak-proof quick-connect couplings.

In yet another aspect, the temperature control unit may reside in a carrier a spaced distance from the socket, while the carrier may be a wheeled carrier or a backpack.

In a further aspect, the thermal exchanger may comprise a water bath wherein a portion of the exchange tube is submerged within the water bath. The water bath may selectively hold hot water to heat the heat exchange fluid in the exchange tube so as to warm the residual limb within the socket or may hold cold water to cool the heat exchange fluid in the exchange tube so as to cool the residual limb within the socket.

In another aspect, the thermal exchanger may comprise a Peltier device wherein a portion of the exchange tube is in communication with the Peltier device. The Peltier device may selectively heat the heat exchange fluid in the exchange tube when the Peltier device is powered by a current in a first direction so as to heat the residual limb within the socket or may cool the heat exchange fluid in the exchange tube when the Peltier device is powered by a current in a second direction so as to cool the residual limb within the socket.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

The accompanying drawings form a part of this specification and are to be read in conjunction therewith, wherein like reference numerals are employed to indicate like parts in the various views, and wherein:

FIG. 1 is a perspective view of an exemplary embodiment of a temperature regulating socket apparatus in use with a prosthetic leg in accordance with an aspect of the present invention;

FIG. 2 is a perspective view of an exemplary embodiment of a temperature regulating socket apparatus in use with a prosthetic arm in accordance with an aspect of the present invention;

FIG. 3 is a transverse cross section view of the embodiment of a temperature regulating socket apparatus shown in FIG. 2, taken generally along line 3-3 in FIG. 2;

FIG. 4 is an expanded lateral cross section view of the embodiment of a temperature regulating socket apparatus shown in FIG. 2, taken generally along line 4-4 in FIG. 2;

FIG. 5 is a partial exploded view of the temperature regulating socket apparatus shown in FIG. 1;

FIG. 6 is a perspective view of an exemplary embodiment of an alternative temperature regulating socket apparatus in accordance with an aspect of the present invention;

FIG. 7 is a perspective view of an exemplary embodiment socket suitable for use within the temperature regulating socket apparatus shown in FIG. 6;

FIG. 7A is a perspective view of another exemplary embodiment socket suitable for use within the temperature regulating socket apparatus shown in FIG. 6;

FIG. 7B is a partial cross section view of the temperature regulating socket apparatus shown in FIG. 7A;

FIG. 8 is a schematic of an exemplary temperature control unit suitable for use within the temperature regulating socket apparatus shown in FIG. 6;

FIG. 9 is a schematic of an alternative exemplary temperature control unit suitable for use within the temperature regulating socket apparatus shown in FIG. 6;

FIG. 10 is a schematic flow diagram showing an exemplary flow path of a heat exchange fluid within the temperature control units shown in FIGS. 8 and 9;

FIG. 11 is a top perspective view of an exemplary combined thermal adjustment element for use within a temperature regulating socket apparatus in accordance with an aspect of the present invention;

FIG. 12 is a partial exploded view of an alternative embodiment of a temperature regulating socket apparatus;

FIG. 13 is a partial cross section view of the alternative embodiment of a temperature regulating socket apparatus shown in FIG. 12; and

FIG. 14 is a cross section view of an exemplary battery pack configured for use with the alternative embodiment of a temperature regulating socket apparatus shown in FIG. 12.

### DETAILED DESCRIPTION OF THE INVENTION

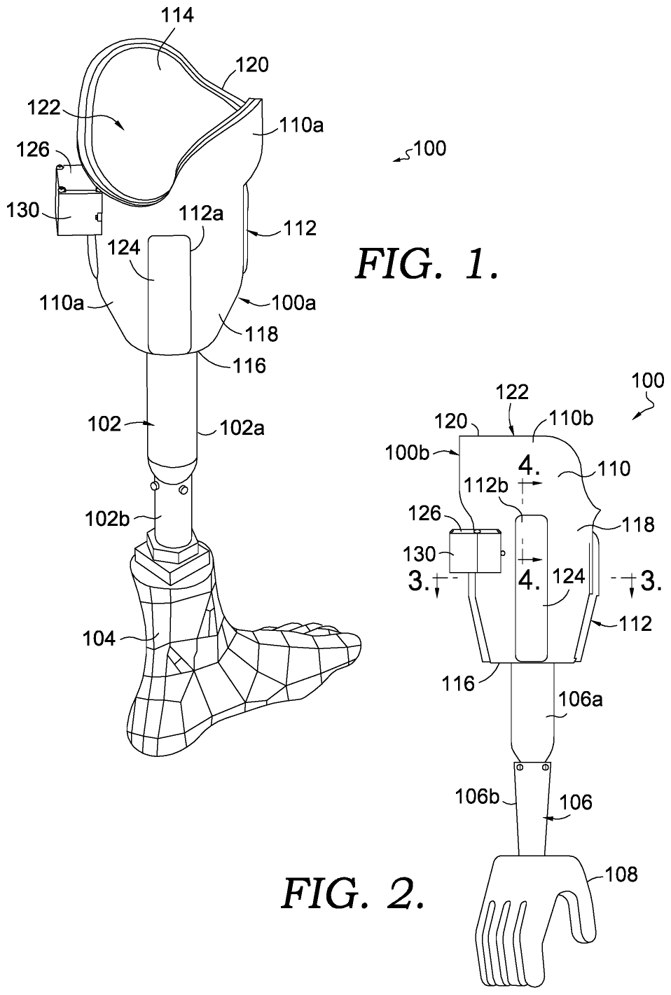

With reference to FIGS. 1-5, exemplary embodiments 100a, 100b of a temperature regulating socket apparatus 100 are shown with a prosthetic leg and foot 102/104 (FIG. 1) and prosthetic arm and hand 106/108 (FIG. 2). Socket apparatus 100 generally includes a socket 110 and a temperature regulating apparatus 112 with the differences between socket apparatuses 100a, 100b directed toward the structural shape of socket 110 (i.e., socket 110a, 110b, respectively) which is configured to receive the amputee's residual limb therein, and, in some cases, the specific layout of the various components of temperature regulating apparatus 112 (i.e., apparatus 112a, 112b) depending upon the size and/or needs of socket 110 and the residual limb. However, the structural fabrication of the socket 100 and general construction of temperature regulating apparatus 112 are similar between socket apparatuses 100a, 100b, as will be discussed in greater detail below.

In accordance with one aspect of the present invention, temperature regulating socket apparatus 100 may be used with a prosthetic liner 114 which may surround or wrap the residual limb an individual who has lost a limb, such as a portion of a leg or arm. Prosthetic liner 114 may be pulled onto the residual limb at the end of a person's leg or arm after an amputation so as to promote circulation within the residual limb, provide heating, and improve comfort when the residual limb is coupled with a prosthetic device. Temperature regulating socket apparatus 100 may also include a mounting element or an upper limb connector portion (upper leg portion 102a, upper arm portion 106a) extending from a distal end 116 of socket 110 while a lower limb connector portion 102b, 106b may be coupled to its respective upper leg portion 102a, upper arm portion 106a. Finally, temperature regulating socket apparatus 100 may include an artificial foot 104 or artificial hand 108 coupled to its respective lower limb connector portion 102b, 106b.

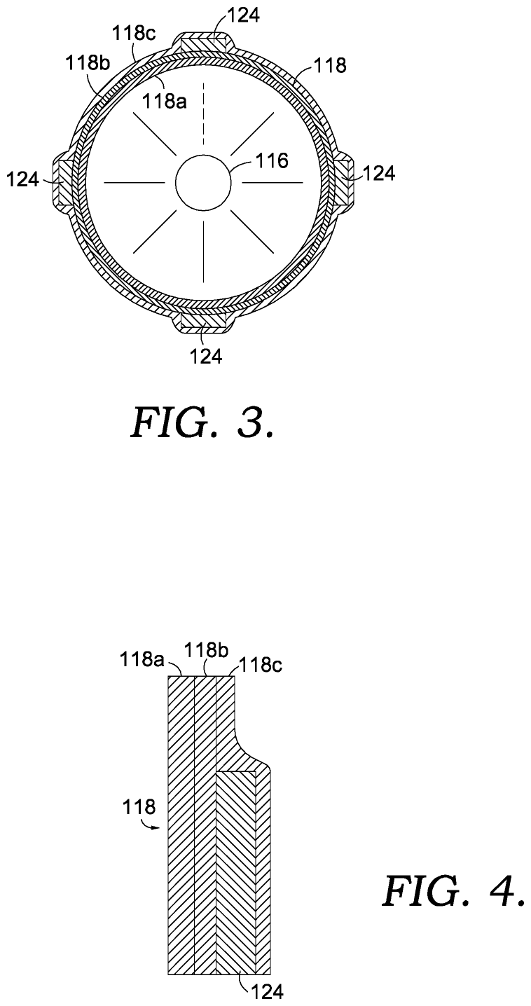

Socket 110 generally includes a continuous side wall 118 having a generally cylindrical configuration having an open brim 120 and closed distal end 116 all defining an interior area 122 between ends 120, 116. Interior area 122 is configured to receive the residual limb (and prosthetic liner 114) therein. In one aspect of the present invention, socket 110 may be cast, such as through use of an epoxy resin. In another aspect and as shown in FIGS. 3 and 4, socket 110 may be manufactured as a laminate (such as layers 118a, 118b, 118c) using materials such as, but not limited to, carbon fiber or fiber glass. In either approach (casting or lamination), the thermal adjustment element 124 of temperature regulating apparatus 112 is embedded within side wall 118 of socket 110.

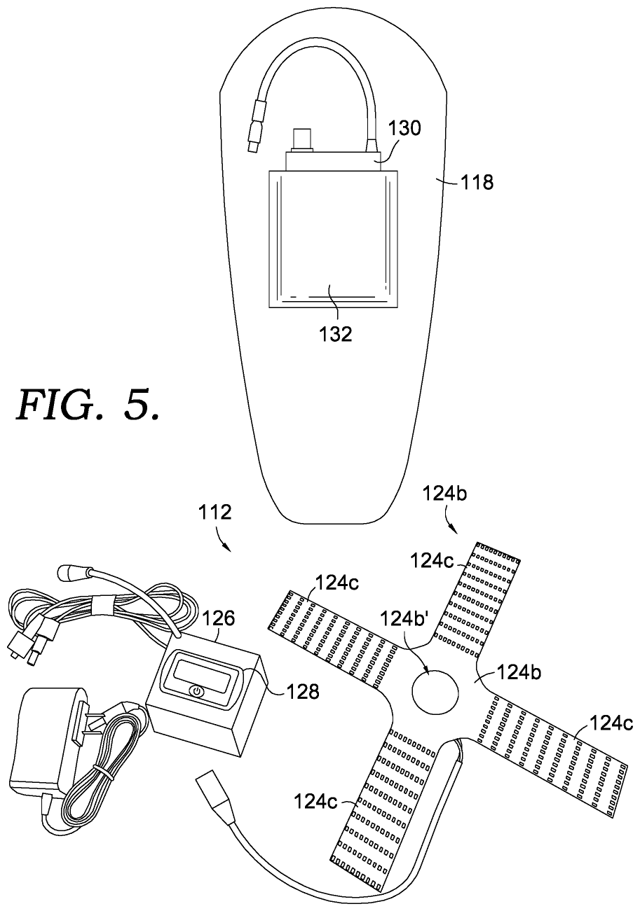

As seen most clearly in FIG. 5, thermal adjustment element 124 may be a flexible heating element 124a. In accordance with an aspect of the present invention, flexible heating element 124a may comprise a base member 124b and a plurality of spaced apart heating units 124c extending outwardly from base member 124b. Base member 124b may be mounted at distal end 116 of socket 110 and may be generally circular with an open interior portion 124b′ configured to allow passage of mounting element (upper leg portion 102a, upper arm portion 106a) therethrough. Heating units 124c may then extend outwardly along side wall 118 from distal end 116 toward open brim 120 of socket 110. Heating units 124c may include a flexible heating element, such as but not limited to a silicon rubber heater, polymer thick film heater or thin film heater, which emits heat when supplied with an electrical current from a power supply 126. In one aspect of the present invention, power supply 126 may be a battery.

A temperature control unit 128 may regulate the current provided to heating units 124c so as to prevent excessive heating of socket 110 and possible injury to the residual limb within interior area 122. Alternatively or additionally, the heating element may also be a positive temperature coefficient (PTC) heater capable of self-regulating so as to output a consistent, user-selected maximum temperature. To prevent overheating and potentially burning a user, the maximum thermal output of heating units 424c may be selected when manufacturing PTC flexible heating units 424c. Battery 126 and temperature control unit 128 may be packaged within a single housing 130. Socket 110 may further include a bracket 132 configured to receive battery 126 and/or housing 130 therein.

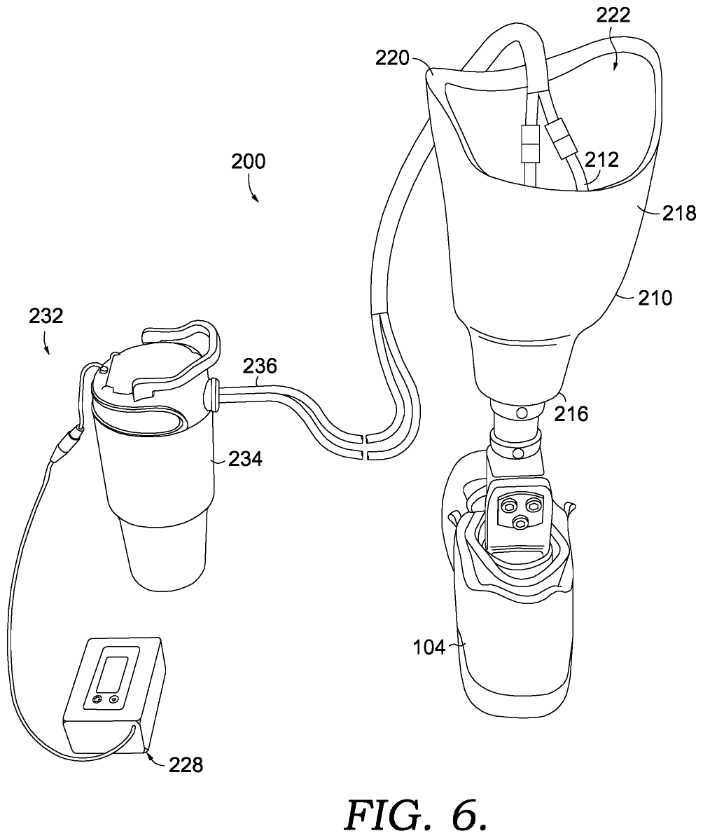

Turning now to FIGS. 6-10, and with particular reference to FIG. 6, in accordance with an aspect of the present invention an exemplary embodiment of an alternative temperature regulating socket apparatus 200 is shown. As shown in FIG. 6, socket apparatus 200 is coupled to an artificial foot 104, although it should be noted that socket apparatus 200 may be configured for use with any type of prosthetic limb device as discussed above. Similar to socket apparatus 100 described above, socket apparatus 200 generally comprises a socket 210 and temperature regulating apparatus 212.

Like socket 110, socket 210 generally includes a continuous side wall 218 having a generally cylindrical configuration having an open brim 220 and closed distal end 216 all defining an interior area 222 between ends 220, 216. Interior area 222 is configured to receive the residual limb (and prosthetic liner 114) therein. In one aspect of the present invention, socket 210 may be cast, such as through use of an epoxy resin. In another aspect, socket 210 may be manufactured as a laminate using materials such as, but not limited to, carbon fiber or fiber glass. In either approach (casting or lamination), the thermal adjustment element 224 of temperature regulating apparatus 212 is embedded within side wall 218 of socket 210.

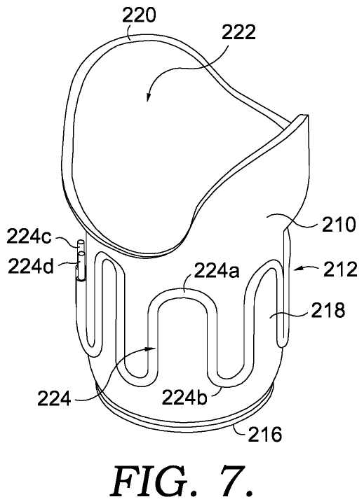

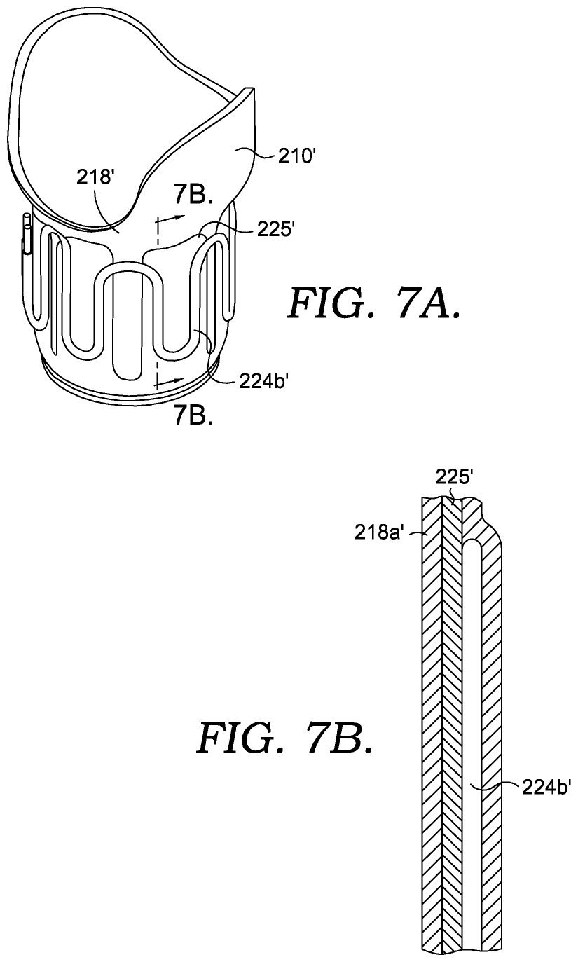

As seen most clearly in FIG. 7, thermal adjustment element 224 may be a flexible heating element 224a comprising a socket tube 224b having opposing inlet and outlet ends 224c, 224d, respectively. In accordance with an aspect of the present invention, socket tube 224b is arranged in a serpentine path about side wall 218 of socket 210, and more specifically has inlet end 224c adjacent outlet end 224d proximate open brim 220. Socket tube 224b may be fabricated of any suitable material, such as but not limited to a metal such as copper or aluminum or a polymeric material such as natural or synthetic rubber, nylon, polyether ether ketone (PEEK), vinyl, cross-linked polyethylene (PEX) or similar material.

As shown in FIGS. 7A and 7B, an alternative socket 210′ is constructed substantially identically to socket 210 shown in FIG. 7 but includes an additional thermal plate element 225′ positioned between all or any portion of socket tube 224b′ and interior laminate layer 218a′ of laminated side wall 218′. In accordance with an aspect of the present invention, thermal plate element 225′ may be a thermally conducting material, such as a metal including, without limitation thereto, aluminum or copper. Thermal plate element 225′ is preferably a lightweight, thin construction (e.g., no more than 3 mm thick) that is suitably malleable to conform to the irregular shape of the socket sidewall 218′. Without being limited to any one theory, thermal plate element 225′ may operate to diffuse the thermal gradient created by a hot or cold heat exchange fluid passing through socket tube 224b′ as will be explained in greater detail below. Thermal plate element 225′ may thus minimize or prevent localized hot or cold spots along the length of socket tube 224b′, thereby improving wearer comfort while also preventing possible burns or frostbite to the wearer's skin.

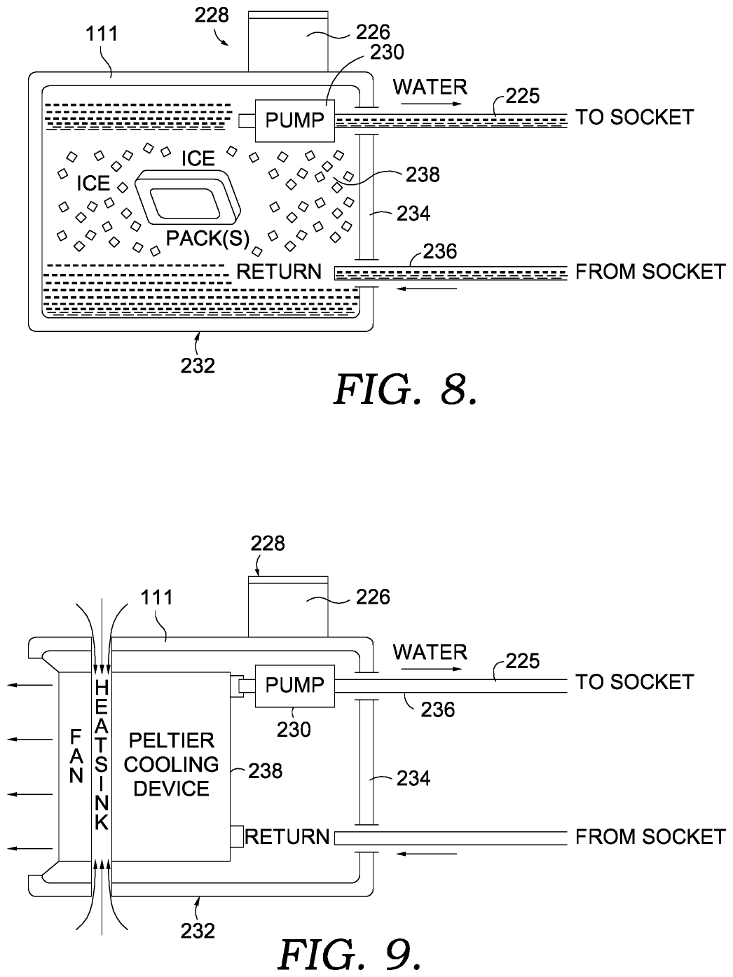

With additional reference to FIGS. 8-10, to provide thermal relief to the amputee, socket tube 224b (or 224b′) is configured to receive and allow flowing passage of a heat exchange fluid 225 therein. In accordance with an aspect of the present invention, heat exchange fluid 225 comprises water. To effectuate flow, temperature control unit 228 may include a pump 230 powered by a power supply 226. As discussed above, power supply 226 may be a battery. In accordance with an aspect of the present invention, temperature control unit 228 may include an optional flow controller 229, such as but not limited to a rheostat or potentiometer, which may regulate the power output from power supply 226 and supplied to pump 230 so as to control the speed of pump 230 and the resultant speed of flow of heat exchange fluid 225 within socket tube 224b (224b′). Temperature control unit 228 may further include a thermal exchanger 232 comprising an exchanger housing 234 and an exchange tube 236. In accordance with an aspect of the present invention, exchanger housing 234 may be a closed vessel configured to receive thermal exchange media 238 therein. By way of example and without limitation thereto, thermal exchange media 238 may comprise one or more of a water bath including ice, one or more ice packs, or one or more Peltier devices (thermoelectric cooler/heater).

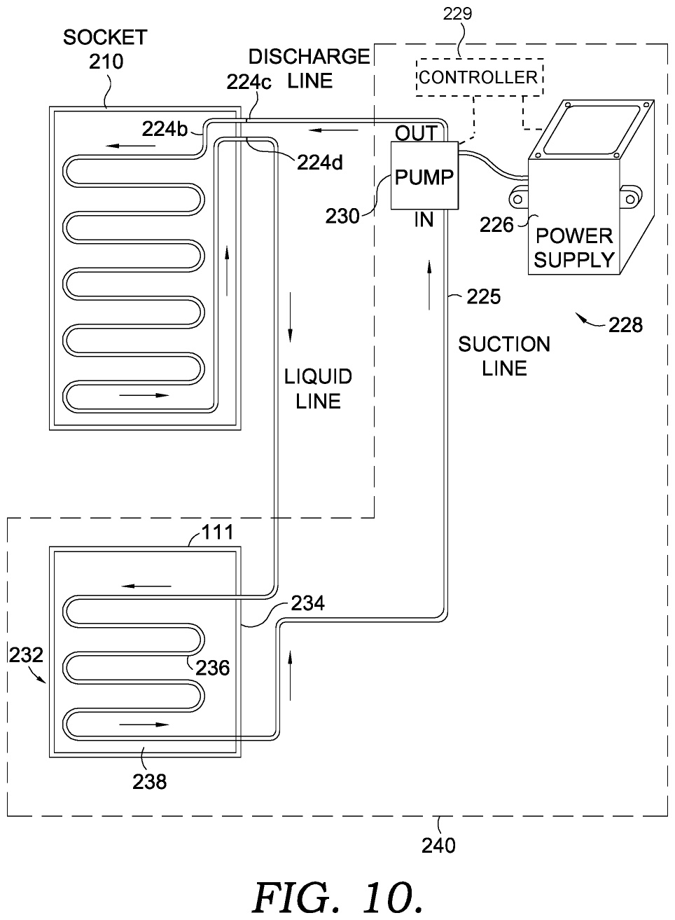

As shown most clearly in FIG. 10, power supply 226 provides electrical power to pump 230 which draws heat exchange fluid 225 from thermal exchanger 232 for delivery to socket tube 224b, 224b′ and socket 210, 210′. In accordance with one aspect of the present invention, the thermal exchange media may include a water bath that selectively holds either a) hot water to heat the heat exchange fluid 225 in exchange tube 236 so as to warm the residual limb within socket 210, 210′; or b) cold water to cool the heat exchange fluid 225 in exchange tube 236 so as to cool the residual limb within socket 210, 210′. Similarly, the Peltier device may selectively a) heat the heat exchange fluid 225 in exchange tube 236 when the Peltier device is powered by a current in a first direction so as to heat the residual limb within socket 210, 210′; or b) cool the heat exchange fluid 225 in exchange tube 236 when the Peltier device is powered by a current in a second direction so as to cool the residual limb within socket 210, 210′. In a further aspect of the present invention, temperature control unit 228 may be configured to reside in a carrier 240 a spaced distance from socket 210, 210′. By way of example and without limitation thereto, carrier 240 may be a wheeled carrier, such as a rolling suitcase or bag, or may be a backpack to be worn by the amputee.

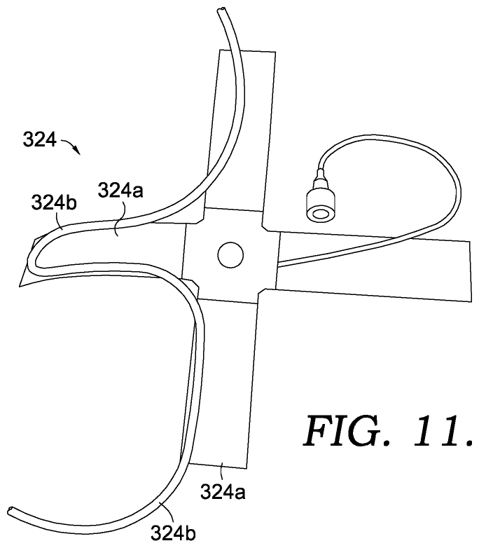

Turning now to FIG. 11, in a further embodiment of a temperature regulating socket apparatus may include a combination thermal adjustment element 324 including both a flexible heating element 324a and a socket tube 324b, the operation of which would be identical to flexible heating element 124a and a socket tube 224b described above.

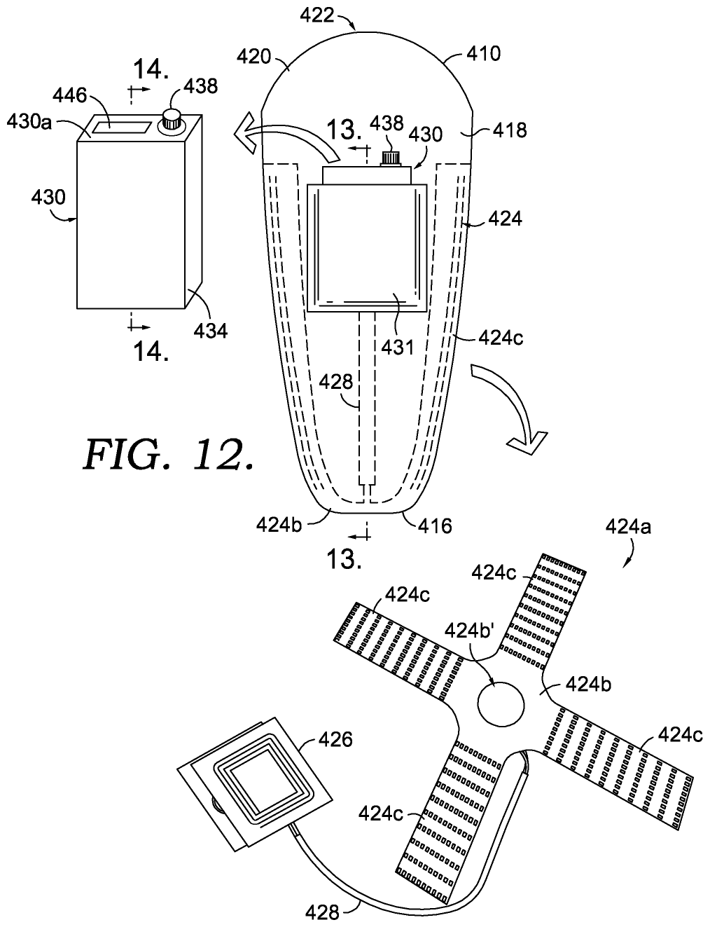

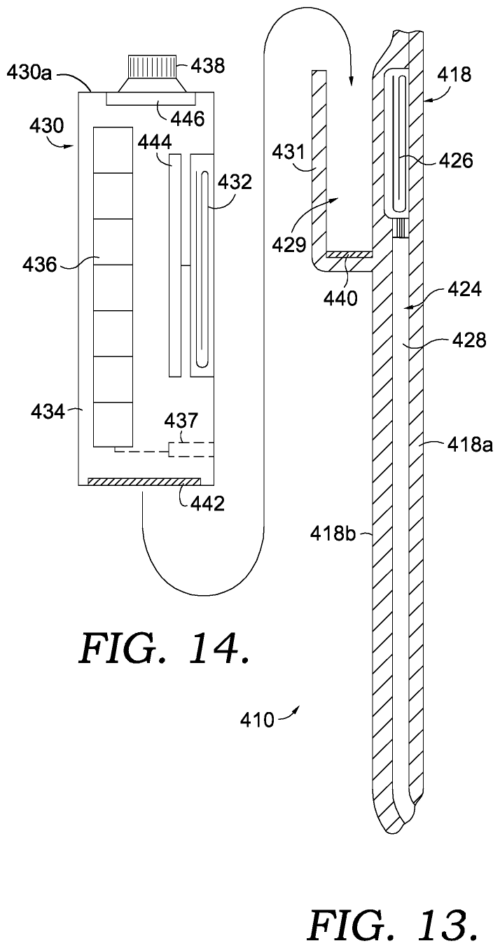

In accordance with another aspect of the present invention, and with specific reference to FIGS. 12-14, an alternative exemplary embodiment of a socket 410 generally includes a continuous side wall 418 having a generally cylindrical configuration having an open brim 420 and closed distal end 416 all defining an interior area 422 between ends 420, 416. Interior area 422 is configured to receive the residual limb (and prosthetic liner 114) therein. In one aspect of the present invention, socket 410 may be cast, such as through use of an epoxy resin. In another aspect and as shown in FIG. 13, socket 410 may be manufactured as a laminate (including at least two laminate layers 418a, 418b) using materials such as, but not limited to, carbon fiber or fiber glass. In either approach (casting or lamination), the thermal adjustment element 424 of temperature regulating apparatus 412 is embedded within side wall 418 of socket 410.

As seen in FIGS. 12-14, thermal adjustment element 424 may be a flexible heating element 424a. In accordance with an aspect of the present invention, flexible heating element 424a may comprise a base member 424b and a plurality of spaced apart heating units 424c extending outwardly from base member 424b. Base member 424b may be mounted at distal end 416 of socket 410 and may be generally circular with an open interior portion 424b′ configured to allow passage of a mounting element (upper leg portion 102a, upper arm portion 106a) therethrough. Heating units 424c may then extend along side wall 418 between inner laminate layer 418a and outer laminate layer 418b and from distal end 416 toward open brim 420 of socket 410.

In one aspect of the present invention, heating units 424c may include a flexible heating element, such as but not limited to a silicon rubber heater, polymer thick film heater or thin film heater, which emits heat when supplied with an electrical current. The heating element may also be a positive temperature coefficient (PTC) heater capable of self-regulating so as to output a consistent user-selected maximum temperature heat. Thermal adjustment element 424 may include a wireless charging receiver coil 426 electrically coupled to flexible heating element 424a, such as via a wired connection 428. As shown most clearly in FIG. 13, wireless charging receiver coil 426 may be positioned between inner/outer laminate layers 418a/418b proximate a pack opening 429 defined by a pack sleeve 431 and outer laminate layer 418b. In one aspect of the present invention, pack sleeve 431 may be integrally formed with socket sidewall 418. As a result, socket 410 may be constructed so as to be waterproof, and more preferably hermetically sealed, such that wireless charging receiver coil 426 and thermal adjustment element 424 can be used in harsh (e.g., rain or snow) without risk of getting wet or suffering an electrical short.

An electric current may be induced within wireless charging receiver coil 426 when a battery pack 430 including a wireless charging transceiver 432 is placed in pack opening 429 in close proximity to wireless charging receiver coil 426, such as via the Qi wireless standard, as will discussed in greater detail below To prevent overheating and potentially burning a user, the maximum thermal output of heating units 424c may be selected when manufacturing PTC flexible heating units 424c.

With continued reference to FIGS. 12-14, battery pack 430 may generally comprise a battery housing 434 which contains one or more batteries 436 arranged therein. Batteries 436 may be electrically coupled to wireless charging transceiver 432 and an ON/OFF switch or button 438. When button 438 is toggled to its ON position, electrical current is supplied to wireless charging transceiver 432 by batteries 436. With wireless charging transceiver 432 properly oriented with wireless charging receiver coil 426, the electrical current passing through wireless charging transceiver 432 may magnetically induce wireless charging receiver coil 426 to produce an electrical current which can then drive thermal output from heating units 424c to warm socket 418 and the residual limb received therein. Battery housing 434 may also be waterproof, and more preferably hermetically sealed, such that the battery pack 430 may be used in conjunction with socket 410 in harsh weather conditions without risk of electrical shorting or shocking the wearer. Heat may still be provided to the wearer through the magnetically induced current without requiring plugs or other connectors which may be susceptible to water intrusion.

In accordance with an aspect of the present invention, batteries 436 are rechargeable batteries, such as lithium ion batteries, which may be recharged by locating battery pack 430 proximate a wireless charging docking station (not shown). The wireless charging docking station may be a dedicated station or may be any suitable commercially available docking station. The wireless charging docking station may then induce a current in wireless charging transceiver 432 which is used to recharge batteries 436. It should be noted that while battery pack 430 has been shown and described as waterproof and with batteries 436 being recharged through magnetic induction, those skilled in the art would understand that other systems and methods for recharging batteries 436 may be used, such as but not limited to plugging battery pack 430 into a standard wall outlet through a USB or micro-USB adapter port 437.

With reference to FIG. 13, a first magnet 440 of a magnet pair may be mounted on pack sleeve 431 or within sidewall 418 proximate pack opening 429. Battery pack 430 may include the second magnet 442 of the magnet pair such that, when properly seated within pack opening 429, first and second magnet pair 440/442 attract one another so as to removably secure battery pack 430 to socket 418 and prevent unwanted dislodging of the battery pack during use. First and second magnet pair 440/442 may also assist in properly aligning wireless charging transceiver 432 with wireless charging receiver coil 426 so as to warm heating units 424c when battery pack 430 is switched ON as described above. Instead of the above-referenced magnet pair, it should be understood that one magnet and a ferromagnetic material may be used to removably secure battery pack 430 in pack opening 429.

In accordance with another aspect of the present invention, battery pack 430 may further include a control board/microcontroller 444 and an indicator 446. Indicator 446 may be located so as to be viewable when battery pack 430 is positioned within pack opening 429, and more preferably is located along a top face 430a of the battery pack 430. In one exemplary embodiment, indicator 446 may comprise a series of light emitting diodes (LEDs) which may represent the charge status of batteries 436. By way of example and without limitation thereto, indicator 446 may include 6 LEDs arranged in a line, where each solid ON indicates ⅙ of the battery charge. Thus, 3 solid ON LEDs and one flashing LED would indicate that the battery charge remaining is between 50-66%, for example. When batteries 436 are being recharged, the number of flashing LEDs may indicate recharge status. As in the above example, 4 flashing LEDs would indicate that the batteries are charging and that the recharging is between 50-66% complete.

In an alternative embodiment, indicator 446 may comprise a display capable of producing alphanumeric characters. By way of example and without limitation thereto, the display may be configured to show a projected battery lifetime, such as by displaying a time expressed as “3 HR 24 MIN” and/or a percentage such as 46% as determined by programmed microcontroller 444 logic. When charging, the display can present a “charging” icon, such as a stylized lightning bolt and/or may display the text “Charging”. In either presentation of battery charging, the display may further present a “percent charged”, such as 56%.

Indicator 446 may further include provisions for presenting the status of the wireless transmission between wireless charging transceiver 432 and wireless charging receiver coil 426, as well as between wireless charging transceiver 432 and the wireless charging docking station. By way of example and without limitation thereto, indicator 446 may include additional LEDs dedicated to indicating transmission. For instance, one or more LEDs may be provided wherein the color status of the LED is indicative of the transmission state. In one example, an LED may be a Blue/Red colored LED whereby when colored Red, there is a fault in the transmission; when colored Blue the transmission is working properly; and when unlit the LED indicates that no receiver has been detected.

The foregoing description of the preferred embodiment of the invention has been presented for the purpose of illustration and description. It is not intended to be exhaustive nor is it intended to limit the invention to the precise form disclosed. It will be apparent to those skilled in the art that the disclosed embodiments may be modified in light of the above teachings. The embodiments described are chosen to provide an illustration of principles of the invention and its practical application to enable thereby one of ordinary skill in the art to utilize the invention in various embodiments and with various modifications as are suited to the particular use contemplated. Therefore, the foregoing description is to be considered exemplary, rather than limiting, and the true scope of the invention is that described in the following claims.

What is claimed is:

1. A temperature regulating socket apparatus for selectively heating or cooling a residual limb of an amputee, the temperature regulating socket apparatus comprising:
a socket having a brim, a distal end and a continuous side wall, all defining an open interior area configured to receive the residual limb therein; and
a temperature regulating apparatus comprising:
a) a thermal adjustment element embedded within the socket side wall;
b) a temperature control unit operatively coupled to the thermal adjustment element; and
c) a power supply operatively coupled to the temperature control unit, wherein when the power supply is in its ON orientation the temperature control unit is powered whereby the temperature control unit regulates the temperature of the thermal adjustment element to selectively heat or cool the residual limb within the socket.

2. The temperature regulating socket apparatus of claim 1 wherein the thermal adjustment element comprises a flexible heating element having a base member configured to be positioned at the distal end of the socket and a plurality of individually spaced apart heating units extending outwardly from the base member, wherein each of the heating elements is configured to extend along a length of the side wall toward the brim.

3. The temperature regulating socket apparatus of claim 2 wherein the temperature control unit selectively adjustably supplies an electric current from the power supply to each of the heating elements.

4. The temperature regulating socket apparatus of claim 2 wherein the base member is dimensioned to encircle a mounting element located at the distal end of the socket, wherein the mounting element is configured to couple a prosthetic device to the socket.

5. The temperature regulating socket apparatus of claim 2 wherein the power supply is a battery, and wherein the battery and the temperature control unit are contained within a common housing.

6. The temperature regulating socket apparatus of claim 5 wherein the socket includes a bracket configured to removably secure the housing to the socket.

7. The temperature regulating socket apparatus of claim 2 wherein the socket is manufactured using casting or lamination.

8. The temperature regulating socket apparatus of claim 7 wherein the lamination uses carbon fiber or fiberglass.

9. The temperature regulating socket apparatus of claim 3 wherein the thermal adjustment element includes a wireless receiving coil, wherein the temperature control unit includes a wireless charging transceiver, and wherein when said wireless charging transceiver is in an ON orientation a current is induced within the wireless receiving coil to supply each of the heating elements.

10. The temperature regulating socket apparatus of claim 3 wherein the wireless receiving coil is hermetically sealed within the socket side wall.

11. The temperature regulating socket apparatus of claim 3 wherein the power supply is a rechargeable battery, and wherein the rechargeable battery and the temperature control unit are contained within a common battery pack housing.

12. The temperature regulating socket apparatus of claim 3 wherein the battery pack housing is hermetically sealed.

13. The temperature regulating socket apparatus of claim 3 wherein the battery pack housing includes a control board and an indicator, wherein the indicator is configured to display one or more of a remaining battery life, a recharging status and a wireless transmission status.

14. The temperature regulating socket apparatus of claim 3 wherein the socket includes a bracket configured to removably secure the battery pack housing to the socket.

15. The temperature regulating socket apparatus of claim 3 wherein the socket includes a pack sleeve configured to removably secure the battery pack housing to the socket.

16. The temperature regulating socket apparatus of claim 15 wherein the pack sleeve and the battery pack housing include respective magnets of a magnet pair to removably secure the battery pack housing in the pack sleeve.

17. The temperature regulating socket apparatus of claim 1 wherein the thermal adjustment element comprises a socket tube having opposing first and second ends.

18. The temperature regulating socket apparatus of claim 17 wherein the socket tube defines a serpentine path about the socket side wall.

19. The temperature regulating socket apparatus of claim 17 wherein the thermal adjustment element further comprises a thermal plate element positioned between the socket tube and the socket side wall facing the open interior area defined by the socket.

20. The temperature regulating socket apparatus of claim 17 wherein the temperature control unit comprises a pump and a thermal exchanger fluidly coupled to the first and second ends of the socket tube, wherein a heat exchange fluid is circulated through the socket tube and the thermal exchanger via the pump.

21. The temperature regulating socket apparatus of claim 20 wherein the heat exchange fluid is water.

22. The temperature regulating socket apparatus of claim 20 wherein the thermal exchanger includes an exchange tube having an inlet end and an outlet end, wherein the inlet end is coupled to the first end of the socket tube via a first coupling and the outlet end is coupled to the second end of the socket tube via a second coupling whereby the socket tube and the exchange tube form a closed loop circuit.

23. The temperature regulating socket apparatus of claim 22 wherein the first and second couplings are leak-proof quick-connect couplings.

24. The temperature regulating socket apparatus of claim 22 wherein the temperature control unit is configured to reside in a carrier located a spaced distance from the socket.

25. The temperature regulating socket apparatus of claim 24 wherein the carrier is a wheeled carrier or a backpack.

26. The temperature regulating socket apparatus of claim 20 wherein the thermal exchanger comprises a water bath wherein a portion of the exchange tube is submerged within the water bath.

27. The temperature regulating socket apparatus of claim 26 wherein the water bath selectively holds either hot water to heat the heat exchange fluid in the exchange tube so as to warm the residual limb within the socket or cold water to cool the heat exchange fluid in the exchange tube so as to cool the residual limb within the socket.
